(19) United States
(12) Patent Application Publication
(10) Pub. No.: US 2011/0033924 A1
(43) Pub. Date: Feb. 10, 2011

(54) Apparatus and Method for Filtering Biological Material

(75) Inventors: Richard George Berry, Aberdeenshire (GB); Edward Alexander Gardyne, Aberdeenshire (GB)

Correspondence Address: MORGAN, LEWIS & BOCKIUS LLP (SF), One Market, Spear Street Tower, Suite 2800, San Francisco, CA, 94105, US

(73) Assignee: Cytosystems Limited, Craibstone, Aberdeen (GB)

(21) Appl. No.: 12/811,995

(22) PCT Filed: Jan. 9, 2009
(86) PCT No.: PCT/GB09/00029

§ 371 Date: Sep. 29, 2010

(30) Foreign Application Priority Data
Jan. 9, 2008 (GB) 0800311.3

(51) Int. Cl. C12M 1/12; C12N 5/09; C12N 5/071

(52) U.S. Cl. 435/325; 435/283.1

(57) ABSTRACT

A method and apparatus for harvesting cells from a fluid so as to preserve the integrity of the cells when used in the diagnosis of cancer. The apparatus comprises a filter adapted to collect cells of a predetermined size, a fluid pathway arranged to transmit fluid to and from the filter, a pump which provide a positive pressure which urges the fluid to the filter along the fluid pathway and a negative pressure which draws the fluid from the filter along the fluid pathway and which operates to pass the sample through the filter in a first direction to collect cells on the filter. A second pump operates to pass a cell preservative fluid through the filter in a second direction to remove cells from the filter for collection. A controller is provided to regulate the flow of fluid along the fluid pathway. The method and apparatus provides a gentle pressure gradient which minimises damage to the cells during the process of filtration and collection.

### FIELD OF THE INVENTION

The present invention relates to an apparatus and method for filtering biological particles and in particular to the filtering of cells from a fluid such as urine to detect bladder cancer.

### BACKGROUND TO THE INVENTION

Human urine typically comprises around 95% water along with urea, a range of ions including ammonium, calcium, sodium, potassium and chloride and other water soluble materials. In addition, cellular material originating from the bladder, kidneys, ureters or urethra is present as a solid suspended in the urine. Consequently, urine may be examined for the presence of abnormal cells which may indicate cancer of the kidney, ureters, bladder, or urethra. In order to do this, a urine, sample is processed in a laboratory and examined under the microscope by a pathologist who looks for the presence of abnormal cells. The current method of processing urine samples to harvest cells for examination involves centrifugation is time consuming, need specialist involvement, requires a number of stages and separate items of equipment.

In the case of bladder cancer, an alternative method called cystoscopy is preferred for determining its presence. However, cystoscopy is invasive and time consuming, both of medical staff time and of laboratory time. Patients who are investigated for suspected bladder cancer in a hospital undergo flexible cystoscopy as an outpatient. Where a positive diagnosis is given, subsequent inpatient investigation is undergone involving general anaesthetic, rigid cystoscopy and early treatment of the potential or actual bladder malignancy. The process is invasive, traumatic and involves a series of repeated cystoscopies for often up to periods of 10 years. It is estimated that somewhere between 16 and 18 cystoscopies will be carried out per patient in that period.

In an effort to reduce the level of invasive intervention, a new diagnostic bladder cell staining technique with a unique antibody based, DNA fixing material is currently being validated. This technology involves a specific antibody fraction known as mini chromosome maintenance antibody (MCM) which selectively targets abnormal epithelial cells in a variety of tissues, including bladder tissue.

This technique will allow a more rapid, more accurate and safer diagnosis at an earlier stage for patients who initially present with symptoms which are suggestive of bladder cancer. It will provide early diagnosis of the condition and may allow the patient to avoid of some of the more intrusive and unpleasant aspects of diagnosis with a management regime which is better for both patient and medical team alike.

This technique would increase the speed and accuracy of diagnosis of bladder cancer, its subsequent recurrence or its subsequent clearance in the ongoing assessment of that individual patient helping avoid the need for both hospital admission and repeated cystoscopies. The potential cost savings arising from such a development are substantial and the emotional and trauma savings to the individual patient are also considerable.

In order to efficiently apply the MCM antibody it is necessary to maximise the number and quality of bladder transitional epithelial cells to which the technique is applied. Therefore, an improved method of harvesting bladder transitional epithelial cells from patient urine samples is desirable.

Prior art has demonstrated that previous attempts to capture bladder transitional epithelial cells using simple filtration techniques have been unsuccessful at least in part because of incorrect choice of filter material, inappropriate process design and the inability to develop a cell harvesting technique which is complementary to and an integrated part of a complete diagnostic protocol.

The use of filters and membranes to selectively isolate bladder transitional epithelial cells from human urine for the purposes of urine cytology has been evaluated in the past without success. Previous studies have concluded that the quality of cells isolated was poor and not appropriate for clinical treatment of urine samples.

Consequently membranes are not used in current urine cytology practice and cells are concentrated using a time consuming technique involving centrifugation, re-suspension of cell pellet, cell washing and preservation.

### SUMMARY OF THE INVENTION

It is an object of the present invention to provide an efficient process and apparatus for harvesting cells from a fluid and in particular harvesting bladder epithelial cells from urine. Another object of the invention is to achieve this is a way that preserves the integrity of the cells so they may be used in the diagnosis of bladder cancer.

In accordance with a first aspect of the invention there is provided an apparatus for collecting cells from a fluid sample, the apparatus comprising:

a filter adapted to collect cells of a predetermined size;
a fluid pathway arranged to transmit fluid to and from the filter; and
pumping means which provides a positive pressure which urges the fluid to the filter along the fluid pathway and a negative pressure which draws the fluid from the filter along the fluid pathway.

Preferably, the pumping means provides a positive pressure by arranging the fluid pathway such that the fluid sample is located above the filter prior to filtration, to create a gravity feed.

Preferably, the pumping means provides a negative pressure by providing a peristaltic pump arranged below the filter to draw the fluid from the filter.

Preferably the filter is adapted to collect bladder epithelial cells from urine.

Preferably, the filter has a pore size of less than 50 microns.

More preferably, the filter has a pore size in the range 1 to 20 microns.

It will be appreciated that the pore size is selected dependant upon the size of cells that are to be collected.

Preferably, the pores are distributed substantially evenly across the surface of the filter.

Preferably, the filter is made from a non-leaching material.

Preferably, the filter is made from polycarbonate.

Preferably, the fluid pathway comprises at least one conduit coupled to at least one sample container and at least one filtrate container.

Preferably, the fluid pathway comprises a cell collector.

Preferably, the fluid pathway comprises a cell preservative container.

Preferably, the conduit comprises, at least in part, tubing that is circumferentially compressible.

Preferably, the conduit is adapted to operate in the peristaltic pump.

Preferably, the tubing is removeably attached in the fluid pathway. In this way, the tubing can be a consumable part of the apparatus which may be replaced each time the apparatus is used, or as often as is necessary.

Preferably, the apparatus further comprises control means adapted to regulate the flow of fluid along the fluid pathway.

Preferably, the control means comprises one or more valves positioned in the fluid pathway to control fluid flow to and from the filter.

Preferably the valves are pinch valves adapted to control the flow of fluid through a pipe in the fluid pathway by compressing the outside of the conduit.

Preferably, the pinch valve is a solenoid pinch valve.

In accordance with a second aspect of the invention there is provided a method for collecting cells from a fluid sample, the method comprising the steps of:

pumping a fluid through a fluid pathway which contains a filter; and
filtering the fluid to collect cells of a predetermined size;
wherein pumping the fluid comprises providing a positive pressure which urges the fluid to the filter along the fluid pathway and a negative pressure which draws the fluid from the filter along the fluid pathway.

Preferably, the positive pressure is provided by arranging the fluid pathway such that the fluid sample is located above the filter prior to filtration, to create a gravity feed.

Preferably, the negative pressure is provided by a peristaltic pump arranged below the filter to draw the fluid from the filter.

Preferably the filter is adapted to collect bladder epithelial cells from urine.

Preferably, the filter has a pore size of less than 50 microns.

More preferably, the filter has a pore size in the range 1 to 20 microns.

It will be appreciated that the pore size is selected dependant upon the size of cells that are to be collected.

Preferably, the pores are distributed substantially evenly across the surface of the filter.

Preferably, the filter is made from a non-leaching material.

Preferably, the filter is made from polycarbonate.

Preferably, the fluid pathway comprises at least one conduit coupled to at least one sample container and at least one filtrate container.

Preferably, the fluid pathway comprises a cell collector.

Preferably, the fluid pathway comprises a cell preservative container.

Preferably, the conduit comprises, at least in part, tubing that is circumferentially compressible.

Preferably, the conduit is adapted to operate in the peristaltic pump.

Preferably, the tubing is removeably attached in the fluid pathway. In this way, the tubing can be a consumable part of the apparatus which may be replaced each time the apparatus is used, or as often as is necessary.

Preferably, fluid flow to and from the filter is controlled by one or more valves positioned in the fluid pathway.

Preferably the valves are pinch valves adapted to control the flow of fluid through a pipe in the fluid pathway by compressing the outside of the conduit.

Preferably, the pinch valve is a solenoid pinch valve.

In accordance with a third aspect of the invention there is provided an apparatus for collecting cells from a fluid sample, the apparatus comprising:

a filter adapted to collect cells of a predetermined size;
a fluid pathway arranged to transmit fluid to and from the filter;
first pumping means which operates to pass the sample through the filter in a first direction to collect cells on the filter;
second pumping means which operates to pass a cell preservative fluid through the filter in a second direction to remove cells from the filter for collection; and
control means adapted to regulate the flow of fluid along the fluid pathway.

Preferably, the first pumping means provides a positive pressure which urges the fluid to the filter along the fluid pathway and a negative pressure which draws the fluid from the filter along the fluid pathway.

Preferably, the first pumping means provides a positive pressure by arranging the fluid pathway such that the fluid sample is located above the filter during filtration, to create a gravity feed.

Preferably, the second pumping means provides a positive pressure which urges fluid to the filter along the fluid pathway and a negative pressure which draws the fluid from the filter along the fluid pathway.

Preferably, the second pumping means provides a positive pressure by arranging the fluid pathway such that the cell preservative fluid is located above the filter after filtration, to create a gravity feed.

Preferably, the first pumping means comprises a peristaltic pump

Preferably, a negative pressure is provided by arranging the peristaltic pump below the filter during filtration to draw the fluid sample from the filter.

Preferably, the second pumping means comprises a peristaltic pump

Preferably, negative pressure is provided by arranging the peristaltic pump below the filter during filtration to draw the fluid sample from the filter.

Preferably the filter is adapted to collect bladder epithelial cells from urine or cells indicative of cancer of the renal system, pelvis, prostate or hyper nephroma.

Preferably, the filter is operatively connected to a vibrator which shakes the filter to assist with the removal of cells from the filter.

Preferably, the filter to vibrate about the plane of the filtration surface of the filter.

Preferably, the filter and the fluid pathway are mounted upon a rotatable platform which positions the fluid sample container above the filter when the first pumping means is in operation and positions a cell preservative fluid container above the filter when the second pumping means is in operation.

Preferably rotation of the rotatable platform is controlled by the control means.

Preferably, the rotatable platform can be oscillated about its axis of rotation.

Preferably, the rotatable platform is powered by a motor such as a stepper motor.

Preferably, the control means comprises one or more valves positioned in the fluid pathway to control fluid flow to and from the filter.

Preferably the valves are pinch valves adapted to control the flow of fluid through a pipe in the fluid pathway by compressing the outside of the conduit.

Preferably, the pinch valve is a solenoid pinch valve.

Preferably, the at least one valve is controllable so as to trap the cell preservative fluid in the filter such that the cells are immersed in the cell collection fluid prior to their further transportation along the fluid pathway.

Preferably, the filter is contained within a vessel, wherein the vessel increases the volume of cell preservative fluid in contact with the filter.

Preferably, at least one valve is closeable such that a head of pressure can be built up when the second pumping means is engaged, said pressure being released upon the opening of the at least one valve in order to assist with the removal of cells from the filter.

Preferably, the control means is programmable.

Preferably, the control means further comprises a microcontroller.

Preferably, the filter has a pore size of less than 50 microns.

More preferably, the filter has a pore size in the range 1 to 20 microns.

It will be appreciated that the pore size is selected dependant upon the size of cells that are to be collected.

Preferably, the pores are distributed substantially evenly across the surface of the filter.

Preferably, the filter is made from a non-leaching material.

Preferably, the filter is made from polycarbonate.

Preferably, the fluid pathway comprises at least one conduit coupled to at least one sample container and at least one filtrate container.

Preferably, the fluid pathway comprises a cell collector.

Preferably, the fluid pathway comprises a cell preservative container.

Preferably, the conduit is comprised of, at least in part, tubing that is circumferentially compressible.

Preferably, the conduit is adapted to operate in the peristaltic pump.

Preferably, the tubing is removeably attached in the fluid pathway. In this way, the tubing can be a consumable part of the apparatus which may be replaced each time the apparatus is used, or as often as is necessary.

In accordance with a fourth aspect of the invention there is provided a method for collecting cells from a fluid sample, the method comprising the steps of:

controllably pumping a fluid through a fluid pathway which contains a filter; and
filtering the fluid to collect cells of a predetermined size;
wherein pumping the fluid comprises
a first stage in which the fluid from the filter is drawn along the fluid pathway to pass the sample through the filter in a first direction to collect cells on the filter; and
a second stage in which a cell preservative fluid is pumped through the filter in a second direction to remove cells from the filter for collection.

Preferably, in the first stage, a positive pressure urges the fluid to the filter along the fluid pathway and a negative pressure draws the fluid away from the filter

Preferably, in the first stage, the positive pressure in the first direction is provided by arranging the fluid pathway such that the fluid sample is located above the filter during filtration, to create a gravity feed.

Preferably, in the second stage, a positive pressure urges the cell preservative fluid to the filter along the fluid pathway and a negative pressure draws the fluid from the filter along the fluid pathway.

Preferably, in the step of pumping the cell preservative fluid, the positive pressure is provided by arranging the fluid pathway such that the cell preservative fluid is located above the filter after filtration, to create a gravity feed.

Preferably, in the first stage, a negative pressure is provided by a peristaltic pump arranged below the filter during filtration to draw the fluid sample from the filter.

Preferably, in the second stage a negative pressure is provided by a peristaltic pump arranged below the filter after filtration to draw the cell preservative fluid from the filter.

Preferably the filter is adapted to collect bladder epithelial cells from urine.

Preferably, the filter is operatively connected to a vibrator which shakes the filter to assist with the removal of cells from the filter.

Preferably, the vibrator causes the filter to vibrate about the plane of the filtration surface of the filter.

Preferably, the filter and the fluid pathway are rotatable in order to position the fluid sample container above the filter during the first stage and positions a cell preservative fluid container above the filter during the second stage.

Preferably, fluid flow through the fluid pathway is provided by one or more valves positioned in the fluid pathway to control fluid flow to and from the filter.

Preferably the valves are pinch valves adapted to control the flow of fluid through a pipe in the fluid pathway by compressing the outside of the conduit.

Preferably, the pinch valve is a solenoid pinch valve.

Preferably, the at least one valve is controllable so as to trap the cell preservative fluid in the filter such that the cells are immersed in the cell collection fluid prior to their further transportation along the fluid pathway.

Preferably, the vessel increases the volume of cell preservative fluid in contact with the filter.

Preferably, at least one valve is closeable such that a head of pressure can be built up when the second pumping means is engaged, said pressure being released upon the opening of the at least one valve in order to assist with the removal of cells from the filter.

Preferably, the control means is programmable.

Preferably, the control means further comprises a microcontroller.

Preferably, the filter has a pore size of less than 50 microns.

More preferably, the filter has a pore size in the range 1 to 20 microns.

Preferably, the pores are distributed substantially evenly across the surface of the filter.

Preferably, the filter is made from a non-leaching material.

Preferably, the filter is made from polycarbonate.

Preferably, the fluid pathway comprises at least one conduit coupled to at least one sample container and at least one filtrate container.

Preferably, the fluid pathway comprises a cell collector.

Preferably, the fluid pathway comprises a cell preservative container.

Preferably, the conduit is comprised of, at least in part, tubing that is circumferentially compressible.

Preferably, the conduit is adapted to operate in the peristaltic pump.

Preferably, the tubing is removeably attached in the fluid pathway. In this way, the tubing can be a consumable part of the apparatus which may be replaced each time the apparatus is used, or as often as is necessary.

In accordance with a fifth aspect of the invention there is provided a method of carrying out an immunoassay, the method comprising the steps of: obtaining a sample comprising cells in a preservative fluid in accordance with the fourth aspect of the invention; and

performing an immunoassay on the sample.

Advantageously, the immunoassay can be carried out on the filtered sample since filtration removes contaminants that would reduce the efficacy of the immunoassay.

### BRIEF DESCRIPTION OF THE DRAWINGS

The present invention will now be described by way of example only with reference to the accompanying drawings in which:

FIG. 1 is schematic diagram of a first embodiment of the present invention;

FIG. 2 is a schematic diagram of a second embodiment of the present invention;

FIG. 3 is an isometric view of the apparatus of the present invention;

FIG. 4 is an exploded isometric view of the apparatus of the present invention; and

FIG. 5 is a layout and operation diagram schematically setting out the process and apparatus of the present invention;

FIG. 6 is a perspective view of another embodiment of an apparatus in accordance with the present invention, the figure showing an extended cylinder and vibration means;

FIG. 7 is a side view of an embodiment of the filter and vibration means in accordance with the present invention;

FIG. 8 is a perspective view of another example of the present invention;

FIG. 9 is an exploded view of the features of the embodiment of the invention shown in FIG. 8;

FIG. 10 is a perspective view of another example of the present invention; and

FIG. 11 is an exploded view of the features of the embodiment of the invention shown in FIG. 10.

### DETAILED DESCRIPTION OF THE DRAWINGS

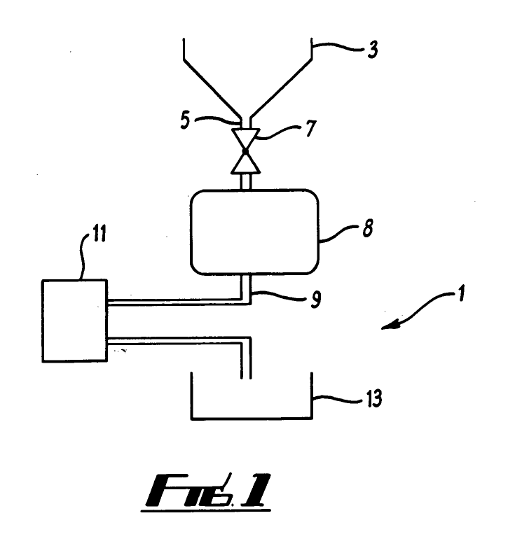

The embodiment of the invention shown in FIG. 1 comprises a sample holder 3 which, in this example, has a conical shape which reduces the amount of liquid sample that will remain in the sample holder 3 making the sample holder easier to clean. The sample holder is connected via a pipe or conduit 5 to valve 7 which controls the flow of liquid from the sample holder 3 to the filter 8. In this example, the filter membrane is a 10 micron polycarbonate disc cell capture filter. Compressible pipe 9 connects the filter 8 to the collector vessel 13 via a peristaltic pump 11.

In use, a urine sample is placed in the sample holder 3 which is positioned above the filter 8 such that gravity acts to force the sample through pipe 5 and into and through the filter 8 when valve 7 is open. The filtration process is enhanced by the action of the peristaltic pump which provides gentle suction below the filter to draw the filtrate through the filter 8. The filter (or membrane) 8 allows smaller cells to pass through into the filtrate collector 13 but retains the bladder transitional epithelial cells on the surface of the membrane. In this embodiment of the invention a single membrane is used to selectively isolate the bladder transitional epithelial cells.

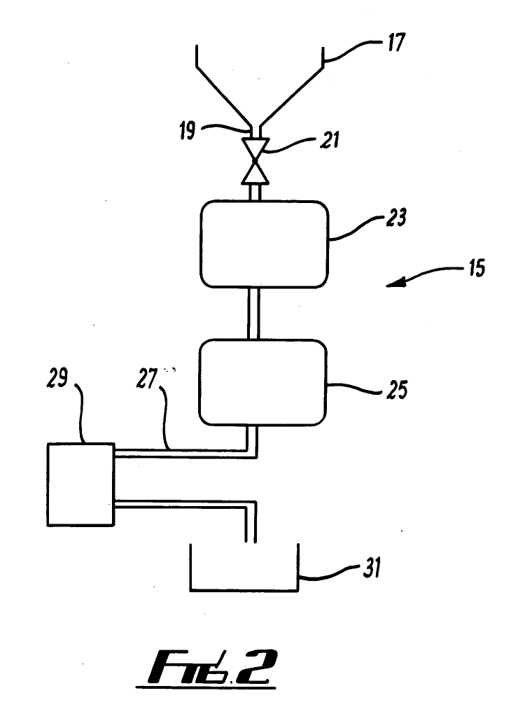

The embodiment of the invention shown in FIG. 2 comprises a conical shaped sample holder 17 connected via a pipe or conduit 19 to valve 21 which controls the flow of liquid from the sample holder 17 to the pre-filter 23. The pre-filter 23 is connected to filter 25 which comprises a 10 micron polycarbonate disc cell capture filter. Compressible pipe 9 connects the filter 8 to the collector vessel 13 via a peristaltic pump 11.

In use, a urine sample is placed in the sample holder 17 which is positioned above the pre-filter 23 and filter 25 such that gravity acts to force the sample through pipe 19 and into and through the pre-filter 23 and filter 25 when valve 19 is open. The filtration process is enhanced by the action of the peristaltic pump which provides gentle suction below the filter to draw the filtrate through the pre-filter 23 and filter 25. In this embodiment of the invention, the pre-filter has a larger pore size to selectively remove larger cells and to break down blood and/or mucous using haemolytic and/or mucolytic agents prior to passage through the main collection membrane. The filter (or membrane) 25 allows smaller cells to pass through into the filtrate collector 31 but retains the bladder transitional epithelial cells on the surface of the membrane.

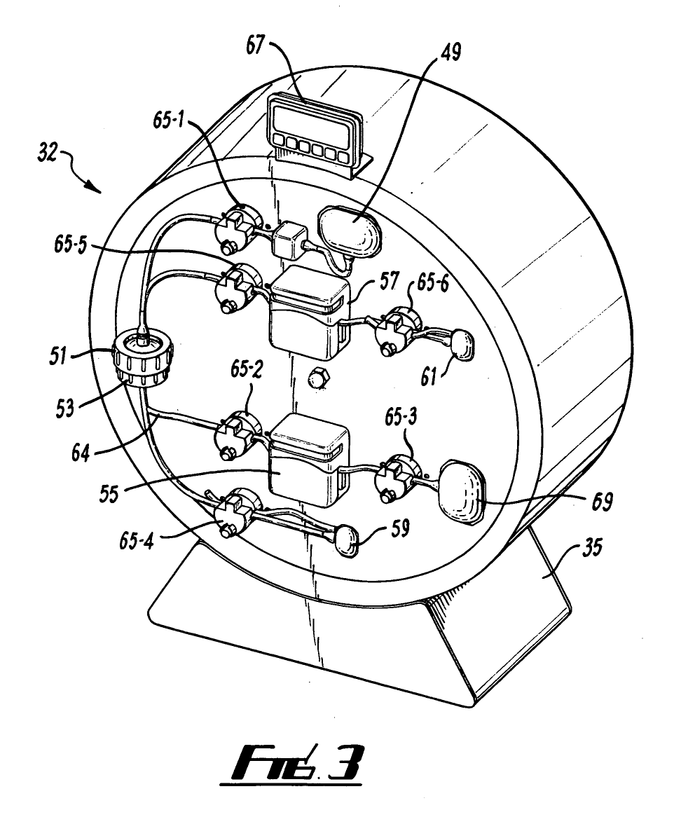

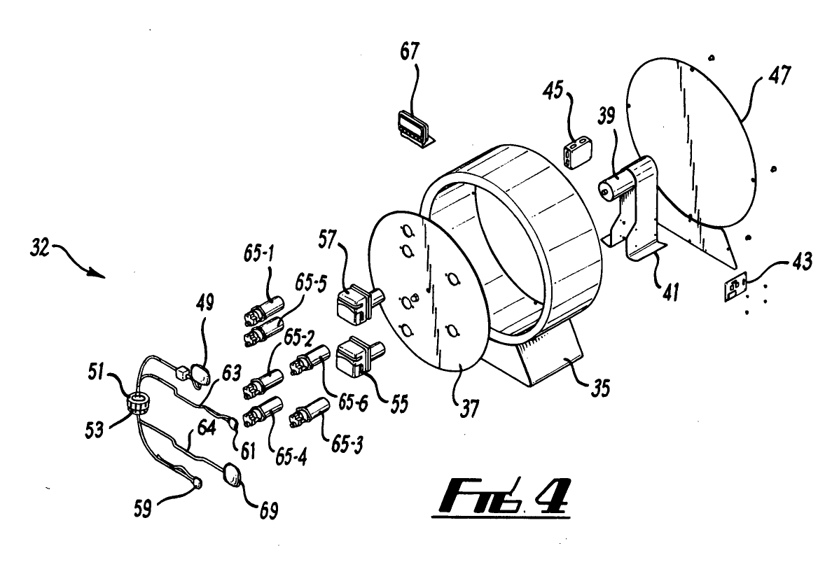

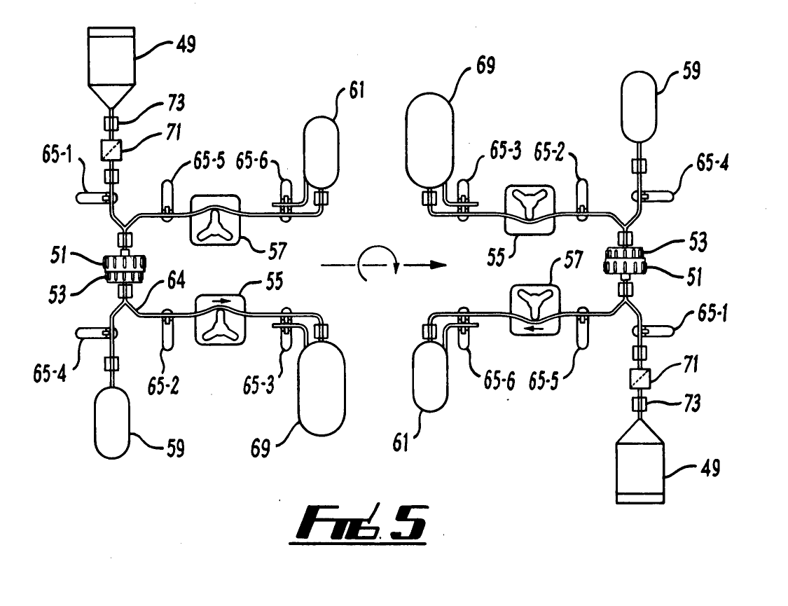

A third embodiment of the present invention is shown in FIG. 3, FIG. 4 and FIG. 5. In this embodiment, an apparatus in accordance with the present invention has been designed as a “desk top” analysis instrument suitable for use in laboratories, clinics, doctors' surgeries or the like.

The instrument comprises a housing 35 on a rotating circular stainless steel face plate 37. A lighter face plate material may also be used such as nylon or polypropylene. A hinged access door (not shown) with viewing window allows access to the cabinet for operation and maintenance. The internal circular face plate 37 is mounted on the shaft of a stepper motor 39 which is mounted on a support structure 41 inside the cabinet housing 35. The stepper motor 39 is controlled by a microprocessor 43 which is also mounted inside the cabinet housing 35.

An electrical junction box 45 is also mounted inside the cabinet housing 35. Internally mounted components such as the microprocessor 43 and the electrical junction box 45 are accessed via a removable access cover 47 on the rear of the cabinet housing 35. The stepper motor 39 is programmed to rotate the face plate by degrees at certain times during the process as described below.

The urine holding cup 49 is manufactured from stainless steel bar stock to ensure there are no welds or recesses that could harbour bacteria. The holding cup has a conical base to ensure that the complete urine sample is pulled from the cup during processing with limited residual film. Stainless steel is chosen for its hygienic qualities, corrosion resistance and its ability to be autoclaved. Polypropylene may be used as an alternative to reduce the weight of the overall assembly. The urine holding cup 49 is orientated at the top of the rotating face plate 37 at the start of the cell collection sequence to provide a gravity head for transfer of the urine through the cell collection membrane 51.

The membrane holder 53 is used to support the polycarbonate cell collection membrane 51. The membrane holder 57 is manufactured from polyfluorocarbon plastic with an inlet and outlet port. The holder is resistant to aggressive chemicals and solvents and can be sterilised in situ using a disinfectant solution of choice and also autoclaved externally if required to ensure sterilisation.

In addition, sterilisation of the apparatus may also be achieved by incorporating an ultra-violet disinfection system inside the cabinet and running a disinfection cycle automatically between processing samples. The ultra-violet disinfection system is not shown in the embodiment of FIGS. 3 to 5 but may be incorporated therein or in another embodiment of the present invention.

Peristaltic pump head 55 is used to pull the urine sample through the cell collection membrane 51 with gravity head assistance depositing the waste urine in a waste container 69 and operates at an appropriate time during the operating sequence controlled by the microprocessor 43. The pump head 55 allows adjustment of settings for tube clamp, tube wall thickness, and tube bore size for easy tube loading.

The benefit of using a peristaltic pump is that the pump is not in direct contact with the urine. This simplifies the sterilisation and maintenance of the unit and eliminates the potential for cross contamination. The urine waste container 69 has a nominal capacity of 200 ml and is manufactured in polythene or polypropylene and is replaced each time the process is executed.

A second peristaltic pump head 57 is used to control reverse-flow through the cell collection membrane 51 and pulls preservative solution stored in a preservative vial 59 through to the cell collection cup 61 at an appropriate time during the operating sequence controlled by microprocessor 43. The cell collection cup is disposable item manufactured in polypropylene or polycarbonate and the preservative can be any suitable preservative such as Preservecyt™ or other methanol or ethanol based preservative, for example.

Fluid is carried around the apparatus using a tube assembly constructed using peristaltic pump tubing 63. The tubing used has a bore diameter of 3.2 mm and a tube wall thickness of 1.6 mm. The tube material is chosen for its compatibility with known constituents of urine including uric acid and also the anticipated disinfection agents that may be used for in situ disinfection of the apparatus. The tube used is manufactured in Marprene®/Bioprene®. There are two pump tube assemblies 63,64 connected to the various items of equipment using quick disconnect Luer type lock couplings allowing the rapid replacement of the tube assemblies when necessary.

The vial 59 containing 20 ml of an appropriate cell preservation fluid is close coupled to the membrane holder 53. The vial 59 is connected to the assembly using a push fit connector and is easy to remove following completion of the cell collection process. The vial 59 is a consumable item and a new vial of preservative fluid is required each time the process is executed.

The urine holding cup 49, waste container 69 and cell collection cup 61 will be secured on the face plate 37 using quick release clips.

Eight solenoid driven pinch valves 65 (65-1-65-8) are used to control fluid movement during the operating sequence under the control of a microprocessor 43. Solenoid pinch valves 65 control fluid flow by pinching the tubing assemblies 63, 64 to close and open the lumen of the tubes with no dead volume. In this way the valves remain contaminant free as no part of the valve is in contact with the urine sample. This greatly simplifies sterilisation of the assembly, preventing cross contamination and allowing easy replacement of the entire fluid path.

The pinch valves 65 use a current passing through a solenoid to induce a magnetic field, which then supplies force to a magnetic plunger. Pinching functionality is achieved when the actuator generates forces that act on a sliding plunger to pinch the tubing. The valves are normally closed or normally open depending on their role in the operating sequence i.e. the tubing is either pinched closed or open when the valves are in a de-energised state. The solenoids are powered using a Direct Current power supply.

An operator interface display with keypad 67 is provided for configuration control of the process. The operator interface display and keypad 67 are connected to the microprocessor 43 and allows the operator to enter sample ID, start the cell harvesting process and change process timers. The display with keypad 67 will also be programmed to prompt the operator to ensure that necessary items have all been connected before the cell harvesting process sequence is run.

FIG. 4 also shows an optional pre-filter 71 and a connection mechanism 73 for easy replacement of the tubing 63, 64 which forms part of the fluid pathway.

An example of the method of using the above apparatus will now be described with reference to FIG. 5.

The cell harvesting process sequence is controlled by the microprocessor 43 as follows.
1. On pressing the key labelled ‘Start Sequence’ the operator interface display 67 prompts the operator to confirm that disinfected (or new) tubing assemblies 63, 64 urine holding cup 49, preservative vial 59, cell collection cup 61 and waste urine container 69 have been connected and a that a fresh membrane (filter) housing and new membrane (filter) 51 has been fitted.
2. Upon operator confirmation via the keypad 67 the microprocessor 43 closes all pinch valves 65 and the operator is asked to place a urine sample in the urine holding cup 49.
3. Upon further operator confirmation via the keypad 67 pinch valves 65-1, 65-2, and 65-3 are opened to open up the flow path from the urine holding cup 49 through the cell collection membrane 51 to the waste urine container 69. Pinch valve 65-3 also vents the waste urine container 69 to allow displacement of enclosed air. Peristaltic pump head 55 is started.
   The pump is run for an adjustable time period (default=3 minutes) to transfer the urine in the holding cup 49 through the cell collection membrane 51 and into the waste urine container 69.
4. The peristaltic pump head 55 is then stopped and the pinch valves 65-1, 65-2, 65-3 are closed.
5. The circular face plate 37 is then rotated 180 degrees via the stepper motor 39.
6. Pinch valve 65-4 is then opened. This valve allows the preservative solution to flow from the preservative vial 59 into the membrane holder 53. Pinch valve 65-5 is positioned to allow the preservative solution to flow through the membrane holder 53 but trap the fluid in the vicinity of the cell collection membrane 51 to ensure that the collected bladder transitional epithelial cells are fully immersed in the preservative and lifted off the surface of the membrane into the solution. The microprocessor 43 also gently rotates the face plate 37 15 degrees from the vertical in both directions to promote movement of the collected cells from the membrane surface into the preservative solution. The rotation sequence lasts for an adjustable time period (default=1 minute)
7. Pinch valves 65-5 and 65-6 are then opened to open the fluid flow path from the membrane holder 53 through to the cell collection cup 61. Pinch valve 65-6 also vents the cell collection cup 61 to allow displacement of enclosed air. Peristaltic pump head 57 is started to pull the preservative solution and suspended epithelial cells into the cell collection cup 61. The pump is run for an adjustable time period (default=1 minute) and the stopped.
8. The cell collection process is now complete and all pinch valves are closed. The operator is then prompted to remove the cell collection cup 61 and acknowledge sequence completion via the keypad.
9. Due to the possibility of cross contamination between successive urine samples both tubing assemblies 63, 64 the urine holding cup 49, membrane housing 53, preservative vial 59 and waste urine container 69 are replaceable each time cells are harvested from a new urine sample.

The cell harvesting sequence as described above may be changed by reprogramming the system to include different steps and change the duration or other characteristics of some of the steps. This may be appropriate when attempting to harvest other types of cell.

All of these components are suitable for autoclaving and may be re-used following approved disinfection procedures. The tubing assemblies 15, 16 may be considered as disposable items. Kits of pre-assembled tubing assemblies will be supplied as consumable spares.

One advantage of the present invention is that it overcomes the cell quality issues at least in part through the novel use of a specific type of membrane to selectively capture the epithelial cells in a gentle process not involving strong centrifugal, vacuum or pumped forces.

In another embodiment of the present invention, the housing is mounted on a fixed face plate with the sample holder positioned above the cell collection filter. In this case, the housing is not inverted and collection of the cells is not gravity assisted and is achieved using a pump only.

In yet another embodiment of the present invention, the housing is mounted on a plate that oscillates to assist in fluid transfer and cell collection.

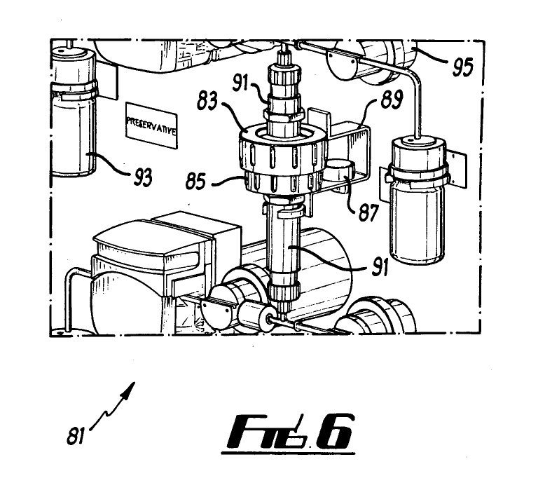

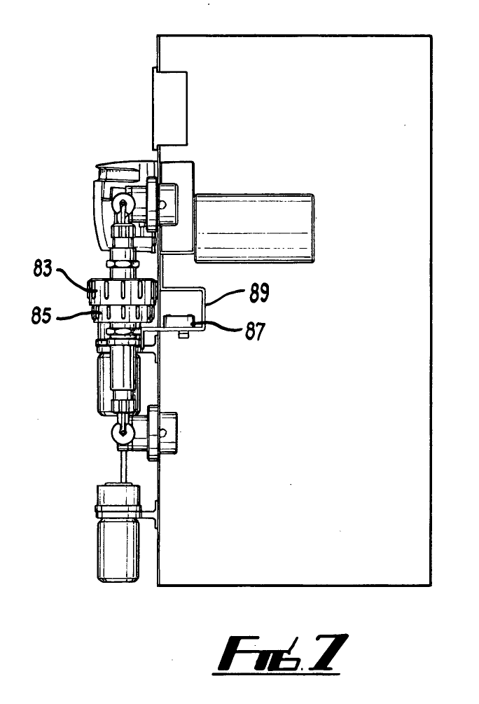

In another embodiment of the present invention the device has been modified in order to increase the recovery of the collected cells from the membrane. This embodiment of the invention is shown in FIGS. 6 and 7.

FIG. 6 shows part of a device 81 in accordance with the present invention which comprises a cell collector 83 coupled to a membrane holder 85. A motor 87 is mounted on a frame 89 which is attached to the cell collector and membrane holder (filter housing). The motor is designed to provide low frequency vibration, typically less than 50 Hz about the plane of the filtration surface of the filter such that the filter is displaced up and down by the vibrations. In this example, a 12V DC eccentric weight vibration motor is used. Cylinder 91 is fitted to one side of the filter housing in order to increase the volume of preservative solution in contact with the cells during cell recovery. Cylinder 91 increases the internal volume capacity of the filter housing from around 4-5 ml to 10 ml in this example. The design of this cylinder can be modified to increase the capacity further however only 20 ml of preservative solution was used, therefore, 10 ml was thought to be a suitable capacity. The cylinder can also be manufactured in polypropylene or some other inert plastic to reduce costs. FIG. 6 also shows the preservative holder 93 and pinch valve 95.

In use, a peristaltic pump (not shown) is used to pump the preservative into the filter housing, through the membrane and extended volume cylinder 91 and out towards the cell collection vessel. This continues for 30 seconds before the vibration motor 87 is activated and the vibration process begins. During the vibration part of the process the pinch valve which is in line to the cell collection vessel (not shown) is closed for 10 seconds allowing fluid to collect in the filter assembly and bathe the cells on the surface of the membrane. Gentle pressure also builds up on the inlet side of the filter housing helping to dislodge the cells. When the valve is closed the vibration motor is activated to provide a low frequency vibration of the filter housing. At the end of the 10 second period the valve on the cell collection line is opened again and the motor is stopped. The pump continues to run for another 30 seconds and then a second vibration sequence is run as described above. Following this the pump continues to run until all the preservative solution has been pumped through into the cell collection vessel. The overall sequence takes around 90 seconds in total.

Several more bursts of pressure pulsing and vibration can be used during the 90 second period to further improve cell collection.

In further embodiments of the present invention, the sequence described above may be changed and the filter housing assembly to optimise the cell collection. In addition, several more bursts of pressure pulsing and vibration may be used during the 90 second period to improve cell collection.

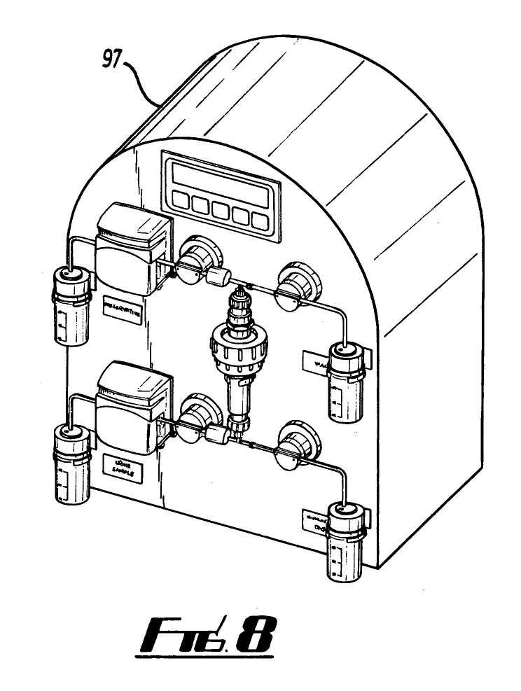

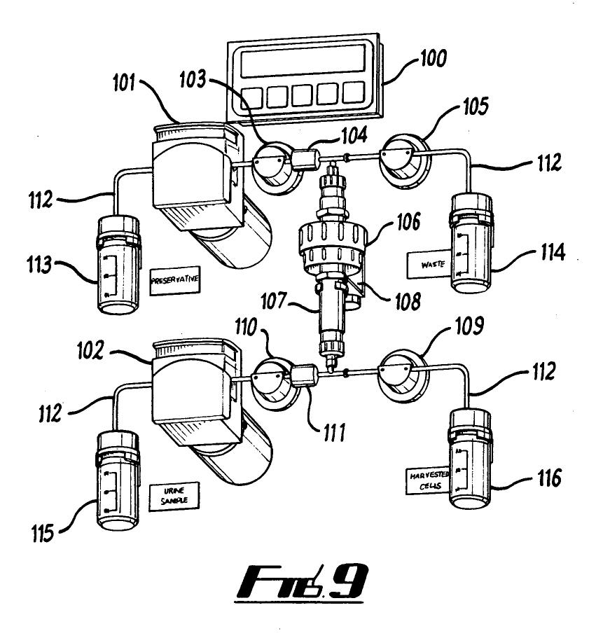

FIGS. 8 and 9 show another embodiment of the present invention in which the two peristaltic pumps are arranged on the fixed fascia of a desk top housing assembly 97 to provide positive pressure pumping. This embodiment provide a faster option for fluid transfer during the cell harvesting sequence because it uses short pump suction paths.

FIG. 9 shows the layout of components in an exploded view of the assembly of FIG. 7. The cell harvesting and cell preservation sequence is under the control of a microprocessor and operator interface 100. Prior to running the cell collection and preservation sequence the operator deposits 20 ml of preservative solution in a vented plastic reservoir bottle 113 and 50 ml of urine in a further vented plastic sample bottle 115 and both bottles are clipped into their respective mounting positions on the housing assembly 97.

Empty vented 50 ml plastic reservoir bottles 114 & 116 are clipped into their respective positions on the housing assembly 97. Also prior to running the cell collection and preservation sequence the operator fits a fresh 8 micron polycarbonate membrane filter installed within a filter holder assembly 106 & 107 to the filter holder shaker clip 108 which is mounted on the housing assembly 97.

Also prior to running the cell collection and preservation sequence the operator fits fresh silicone tubing suitable for use with peristaltic pumps 101 & 102 and pinch valves 103, 105, 109, 110 to interconnect the plastic reservoir bottles 113, 114, 115 & 116 and filter holder assembly 106 & 107 using leur type fittings. The tube assembly incorporates check valves 104 & 111

When the components have been assembled as described and shown in FIG. 9 the operator initiates the cell collection and preservation sequence via the operator interface 100 and the sequence proceeds under automatic control via a microprocessor. On initiation the microprocessor immediately closes pinch valve 103 and pinch valve 109. When the valves are closed the cell harvesting pump 102 is started and runs for a cell collection period configurable via the operator interface 100. Different settings may be chosen based on the viscosity and general quality of the urine sample. A typical time period is 180 seconds but could be longer or shorter

When the cell harvesting pump 102 is running the urine sample is drawn from the vented sample vessel 115 through the silicone tubing assembly 112, through open pinch valve 110, through check valve 111, through the filter housing extension 107, through the 8 micron membrane situated in the filter holder 106, through open pinch valve 105, through the silicon tubing assembly 112 and into the vented waste vessel 114. As the urine passes through the 8 micron membrane, bladder epithelial cells are collected on the lower surface of the membrane.

When the cell collection period has expired, the microprocessor immediately stops the cell collection pump 102 and closes pinch valves 110 and 105. Pinch valves 103 and 109 are then opened. When the valves are open the cell preservation pump 101 is started and runs for a period programmed into the microprocessor memory. This period is typically 120 seconds but may be more or less.

When the cell preservation pump 101 is running the preservative solution is drawn from the vented storage vessel 113 through the silicone tubing assembly 112, through open pinch valve 103, through check valve 104, through the 8 micron membrane situated in the filter holder 106, through the filter housing extension 107, through open pinch valve 109, through the silicone tubing assembly 112 and into the vented cell collection vessel 116.

Under the control of the microprocessor, 15-30 seconds after the cell preservation pump has been started, pinch valve 109 is closed for a short period typically 5-10 seconds and the filter holder 106 & 107 is shaken for the same short period via the filter holder clip 108 using an eccentric motor integrated within the clip as shown in FIG. 7.

With pinch valve 109 closed and the cell preservation pump 101 still running, the preservative fluid collects in the filter housing extension 107 building up pressure inside the extension and flooding the cells which were collected on the lower surface of the membrane. The vibration caused by the shaking action dislodges the collected cells from the membrane surface and into the preservative solution inside 107.

Following the pre programmed period, pinch valve 109 is open again to release the pressure as a pulse and flush the retained preservative solution through the fluid path as described.

During the cell preservation sequence the microprocessor may repeat the pressure build up and shaking sequence several times to maximise recovery of cells from the membrane. This is configurable via the operator interface 100

When the cell preservation period has expired, the microprocessor immediately stops the preservation pump 101 and closes pinch valves 103 and 109. This signifies the end of the sequence. At this point the harvested cells have been collected and preserved in the vented cell collection bottle 116.

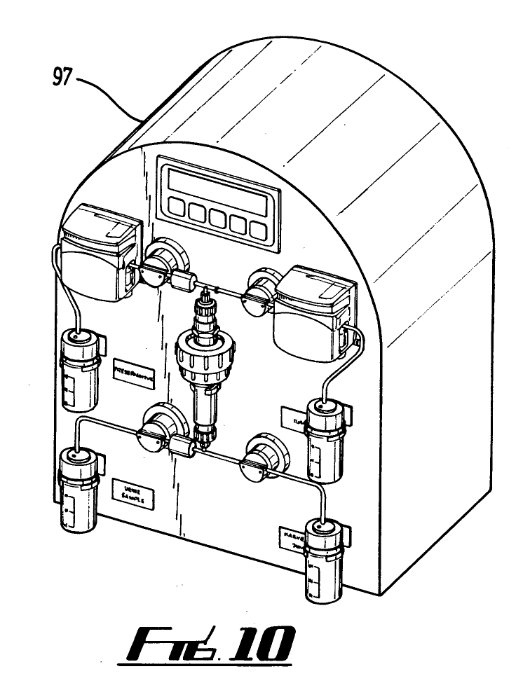

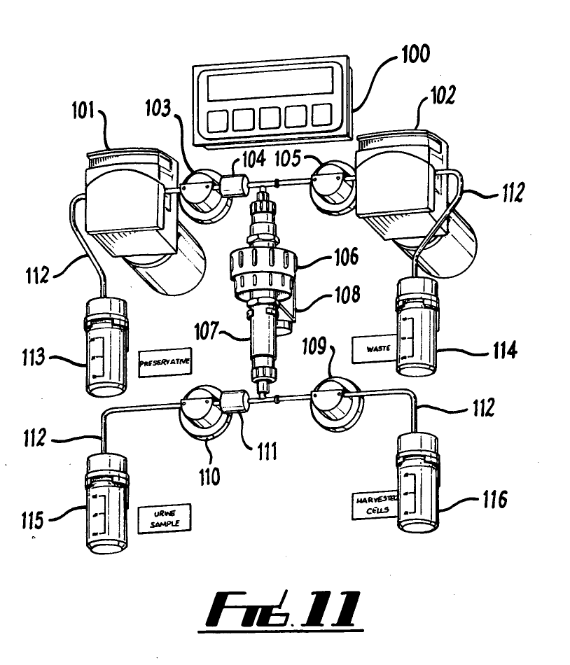

In another embodiment of the present invention as shown in FIGS. 10 and 11, the two peristaltic pumps are arranged on the fixed fascia of a desk top housing assembly 97 to provide positive pressure pumping and suction.

This embodiment differs from the embodiment described with reference to FIGS. 8 and 9 as it provides a more gentle option for cell collection using the suction force developed by the cell harvesting pump 102 to draw the urine through the membrane in a less forceful manner reducing the possibility that the collected cells will stick in the lumen of the filter pores. This option extends the cell collection time as the fluid transfer takes longer.

FIG. 10 shows the layout of components in an exploded view of the assembly. The cell harvesting and cell preservation sequence is under the control of a microprocessor and operator interface 100

Prior to running the cell collection and preservation sequence the operator deposits 20 ml of preservative solution in a vented plastic reservoir bottle 113 and 50 ml of urine in a further vented plastic sample bottle 115 and both bottles are clipped into their respective mounting positions on the housing assembly 97

Empty vented 50 ml plastic reservoir bottles 114 & 116 are clipped into their respective positions on the housing assembly 97

Also prior to running the cell collection and preservation sequence the operator fits a fresh 8 micron polycarbonate membrane filter installed within a filter holder assembly 106 & 107 to the filter holder shaker clip 108 which is mounted on the housing assembly 97.

Also prior to running the cell collection and preservation sequence the operator fits fresh silicone tubing suitable for use with peristaltic pumps 101 & 102 and pinch valves 103, 105, 109, 110 to interconnect the plastic reservoir bottles 113, 114, 115 & 116 and filter holder assembly 106 & 107 using leur type fittings. The tube assembly incorporates check valves 104 & 111

When the components have been assembled as described and shown on FIG. 10 the operator initiates the cell collection and preservation sequence via the operator interface 100 and the sequence proceeds under automatic control via a microprocessor.

On initiation the microprocessor immediately closes pinch valve 103 and pinch valve 109.

When the valves are closed the cell harvesting pump 102 is started and runs for a cell collection period configurable via the operator interface 100. Different settings may be chosen based on the viscosity and general quality of the urine sample. A typical time period is 180 seconds but could be longer or shorter.

When the cell harvesting pump, 102 is running the urine sample is drawn from the vented sample vessel 115 through the silicone tubing assembly 112, through the cell harvesting pump 102, through open pinch valve 110, through check valve 111, through the filter housing extension 107, through the 8 micron membrane situated in the filter holder 106, through open pinch valve 105, through cell harvesting pump 102, through the silicon tubing assembly 112 and into the vented waste vessel 114. As the urine passes through the 8 micron membrane bladder epithelial cells are collected on the lower surface of the membrane.

When the cell collection period has expired, the microprocessor immediately stops the cell collection pump 102 and closes pinch valves 110 and 105. Pinch valves 103 and 109 are then opened.

When the valves are open the cell preservation pump 101 is started and runs for a period programmed into the microprocessor memory. This period is typically 120 seconds but may be more or less.

When the cell preservation pump 101 is running the preservative solution is drawn from the vented storage vessel 113 through the silicone tubing assembly 112, through open pinch valve 103, through check valve 104, through the 8 micron membrane situated in the filter holder 106, through the filter housing extension 107, through open pinch valve 109, through the silicone tubing assembly 112 and into the vented cell collection vessel 116.

Under the control of the microprocessor, 15-30 seconds after the cell preservation pump has been started, pinch valve 109 is closed for a short period typically 5-10 seconds and the filter holder 106 & 107 is shaken for the same short period via the filter holder clip 108 using an eccentric motor integrated within the clip.

With pinch valve 109 closed and the cell preservation pump 101 still running, the preservative fluid collects in the filter housing extension 107 building up pressure across the membrane and flooding the cells which were collected on the lower surface of the membrane. The vibration caused by the shaking action acts to dislodge the collected cells from the membrane surface and into the preservative solution inside 107.

Following the pre programmed short period, pinch valve 109 is open again to release the pressure as a pulse and flush the retained preservative solution through the fluid path as described. During the cell preservation sequence the microprocessor may repeat the pressure build up and shaking sequence several times to maximise recovery of cells from the membrane. This is configurable via the operator interface 100.

When the cell preservation period has expired, the microprocessor immediately stops the preservation pump 101 and closes pinch valves 103 and 109.

This signifies the end of the sequence. At this point the harvested cells have been collected and preserved in the vented cell collection bottle 116.

In the embodiments of the present invention described above, urine samples can be processed to provide cells which are harvested and fixed using a fixative soon after the urine has been voided by the patient. Once fixed, the cells may exist for a period determined largely by the quality of the fixative, typically for a period of up to 30 days. The present invention thereby avoids the cell damage caused by retaining the cells in the urine sample, freezing the urine sample (which can reduce the cell half life to 3-5 hours) and provides a means for harvesting cells in a hospital clinic or other suitable location before the sample is sent to be tested. The present invention also speeds up the processing of urine samples allowing faster implementation of the bladder cancer diagnostic techniques used in routine urine cytology.

### Experimental Data

An initial comparative study was undertaken to determine the effectiveness, reliability and reproducibility of the present invention when compared with standard laboratory based centrifugation in the preparation of a liquid based cell slide (LBC) for the determination of the shape, character and integrity of epithelial bladder cells in man.

This study measured the effectiveness, reliability and reproducibility of an embodiment of the device of the present invention for use in the preparation of epithelial cells lining the urinary bladder as part of the process for determining the presence or otherwise of Transitional Cell Carcinoma (TCC) of the bladder in man.

The quantity and quality of cell content captured by the device of the present invention were compared with standard best laboratory practice in the form of routine centrifugation of paired split samples of freshly voided urines from the same patient.

### Methodology

Normal volunteers who had no urinary symptoms (that is no symptoms or signs suggestive of renal disease) and patients who had gross haematuria, microscopic haematuria or referred to the Haematuria Clinic with suspected bladder cancer as a presenting symptom were enrolled in the study. In addition, patients who were returning for repeat TCC evaluation and surveillance of known bladder cancer were also selected for the study. All volunteers and patients agreed to evaluation of their urine cellular content under the present study circumstances (standard cytology practice). Volunteers and patients were selected on the ability to pass at least 100 ml of urine (or larger volumes) in one passage following the admission to the Ward or Outpatient Department.

Patients involved in the study to evaluate MCM antibody as a bladder cancer diagnostic agent were selected and of the 100 ml samples passed or more, 50 mls of the sample was mixed (inverted) in the laboratory and split randomly for cell collection either through a device according to the present invention, or in a centrifuge system. Cells from both methods were then resuspended, mounted and stained on a slide using Liquid Based Cytology.

LBC and processing of samples using a device in accordance with the present invention are compared. 11 specimens had completed pairs of total and malignant cell counts.

Early data on 20 such patients indicated that there were no significant differences in either cell quality or content when examined by a Consultant Cytopathologist and there were indications that the cellular content and nuclear content in cells captured by the device of the present invention appeared somewhat better and clearer in definition under examination with a microscope. Further, initial data on the epithelial bladder cells indicated that where these cells were washed off into Preservcyt solution, then cellular and nuclear integrity were maintained for a minimum period of 15 days. It is expected that studies will confirm longevity and integrity of cellular/nuclear structures for around 30 days. Percentage malignant cells were calculated for each.

It can be seen that the device of the present invention harvested an overall higher percentage of malignant cells than LBC for same specimen, although the majority are very similar or identical.

TABLE 1
Matched Pairs t-test for % Malignant Cells paired by specimen
(Tests difference of pairs has a zero mean) which shows a significant
positive correlation between pairs (Sig. < 0.0005)
Paired Samples Statistics

| | | | | | Std. Error |
|---|---|---|---|---|---|
| | | Mean | N | Std. Deviation | Mean |
| Pair 1 | lbc | 75.5455 | 11 | 32.40483 | 9.77042 |
| | filter | 81.3838 | 11 | 27.85768 | 8.39941 |

Paired Samples Correlations

| | | N | Correlation | Sig. |
|---|---|---|---|---|
| Pair 1 | lbc & filter | 11 | .941 | .000 |

TABLE 2
Paired Samples Test shows no evidence of mean of differences differing from zero (Sig. = 0.118)
Paired Samples Test

| | Paired Differences | | | | | | | |
|---|---|---|---|---|---|---|---|---|
| | | | | 95% Confidence | | | | |
| | | | | Interval of the | | | | |
| | | | Std. Error | Difference | | | | |
| | Mean | Std. Deviation | Mean | Lower | Upper | t | df | Sig. (2-tailed) |
| Pair 1 lbc - filter | −5.8384 | 11.31593 | 3.41188 | −13.4405 | 1.7638 | −1.711 | 10 | .118 |

TABLE 3
Wilcoxon Signed Ranks Test (Tests for zero median
for differences) shows no evidence of median of
differences differing from zero (Sig. = 0.123).

| | Ranks | | | |
|---|---|---|---|---|
| | | N | Mean Rank | Sum of Ranks |
| filter - lbc | Negative Ranks | 2^{a} | 3.50 | 7.00 |
| | Positive Ranks | 6^{b} | 4.83 | 29.00 |
| | Ties | 3^{c} | | |
| | Total | 11 | | |

^{a}filter < lbc
^{b}filter > lbc
^{c}lbc = filter

Test Statistics^{b}

| | filter - lbc |
|---|---|
| Z | −1.540^{a} |
| Asymp. Sig. (2-tailed) | .123 |

^{a}Based on negative ranks.
^{b}Wilcoxon Signed Ranks Test

TABLE 4
No evidence of Filter results being higher than LBC (Sig. = 0.289)
Test Statistics^{b}

| | filter - lbc |
|---|---|
| Exact Sig. (2-tailed) | .289^{a} |

^{a}Binomial distribution used.
^{b}Sign Test

| Frequencies | | N |
|---|---|---|
| filter - lbc | Negative Differences^{a} | 2 |
| | Positive Differences^{b} | 6 |
| | Ties^{c} | 3 |
| | Total | 11 |

^{a}filter < lbc
^{b}filter> lbc
^{c}lbc = filter

### 6 Tables for Diagnosis by Specimen

| | | Diagnosis | | | |
|---|---|---|---|---|---|
| | | atypia | malignant | neg | US |
| | | Count | Count | Count | Count |
| 62/01/03 | Filter | | 1 | | |
| | LBC | | 1 | | |
| 63/01/01 | Filter | | 1 | | |
| | LBC | | 1 | | |
| 63/01/02 | Filter | | 1 | | |
| | LBC | | 1 | | |
| 63/01/03 | Filter | | 1 | | |
| | LBC | | 1 | | |
| 64/01/01 | Filter | | 1 | | |
| | LBC | | 1 | | |
| 64/01/02 | Filter | | 1 | | |
| | LBC | | 1 | | |
| 65/01/01 | Filter | | | | 1 |
| | LBC | | | 1 | |
| 65/01/02 | Filter | | | 1 | |
| | LBC | | | 1 | |
| 65/01/03 | Filter | | | 1 | |
| | LBC | | | 1 | |
| 67/01/01 | Filter | | 1 | | |
| | LBC | | 1 | | |
| 68/01/01 | Filter | | 1 | | |
| | LBC | | 1 | | |
| 70/01/02 | Filter | 1 | | | |
| | LBC | 1 | | | |
| 70/01/03 | Filter | 1 | | | |
| | LBC | 1 | | | |
| 70/01/04 | Filter | | 1 | | |
| | LBC | | 1 | | |
| 71/01/01 | Filter | | 1 | | |
| | LBC | | 1 | | |
| 72/01/01 | Filter | | | | 1 |
| | LBC | | | | 1 |
| 74/01/01 | Filter | | 1 | | |
| | LBC | | 1 | | |
| 75/01/01 | Filter | 1 | | | |
| | LBC | 1 | | | |
| 76/01/01 | Filter | | 1 | | |
| | LBC | | 1 | | |

Only Specimen 65/01/01 differs in diagnosis

The method of the present invention can selectively isolate bladder transitional epithelial cells from urine samples (preferably fresh or recently voided samples) and automatically preserve the cells in a proprietary preservative solution. In this form the preserved cells can be coated on a slide as a mono layer using an existing thin-layer processing technique allowing application of the diagnostic antibody. The test can also collect cells that allow the detection of a range of cancers related to the uro-epthelial tract and will include specifically cancer of the ureter, cancer of the renal pelvis, bladder cancer, prostate cancer, and hyper nephroma. Semen can be used as an analyte for the detection of prostate cancer.

Improvements and modifications may be incorporated herein without deviating from the scope of the invention.

1. An apparatus for collecting cells from a fluid sample, the apparatus comprising:
a filter adapted to collect cells of a predetermined size;
a fluid pathway arranged to transmit fluid to and from the filter;
first pumping means which operates to pass the sample through the filter in a first direction to collect cells on the filter;
second pumping means which operates to pass a cell preservative fluid through the filter in a second direction to remove cells from the filter for collection; and
control means adapted to regulate the flow of fluid along the fluid pathway.

2. An apparatus as claimed in claim 1 wherein the first pumping means provides a positive pressure which urges the fluid to the filter along the fluid pathway and a negative pressure which draws the fluid from the filter along the fluid pathway.

3. An apparatus as claimed in claim 2 wherein, the first pumping means provides a positive pressure by arranging the fluid pathway such that the fluid sample is located above the filter during filtration, to create a gravity feed.

4. An apparatus as claimed in claim 1 wherein, the second pumping means provides a positive pressure which urges the cell preservative fluid to the filter along the fluid pathway and a negative pressure which draws the cell preservative fluid from the filter along the fluid pathway.

5. An apparatus as claimed in claim 4 wherein, the second pumping means provides a positive pressure by arranging the fluid pathway such that the cell preservative fluid is located above the filter after filtration, to create a gravity feed.

6. An apparatus as claimed in claim 1 wherein, the first pumping means comprises a peristaltic pump,

7. An apparatus as claimed in claim 6 wherein a negative pressure is provided by arranging the peristaltic pump below the filter during filtration to draw the fluid sample from the filter.

8. An apparatus as claimed in claim 1 wherein the second pumping means comprises a peristaltic pump.

9. An apparatus as claimed in claim 8 wherein negative pressure is provided by arranging the peristaltic pump below the filter during filtration to draw the fluid sample from the filter.

10. An apparatus as claimed in claim 1 wherein the filter is adapted to collect bladder epithelial cells from urine, or cells indicative of cancer of the renal system, pelvis, prostate or hyper nephroma.

11. An apparatus as claimed in claim 1 wherein the filter is operatively connected to a vibrator which shakes the filter to assist with the removal of cells from the filter.

12. An apparatus as claimed in claim 11 wherein the vibrator causes the filter to vibrate about the plane of the filtration surface of the filter.

13. An apparatus as claimed in claim 1 wherein, the filter and the fluid pathway are mounted upon a rotatable platform which positions the fluid sample container above the filter when the first pumping means is in operation and positions a cell preservative fluid container above the filter when the second pumping means is in operation.

14. An apparatus as claimed in claim 13 wherein, rotation of the rotatable platform is controlled by the control means.

15. An apparatus as claimed in claim 13 wherein, the rotatable platform can be oscillated about its axis of rotation.

16. An apparatus as claimed in claim 1 wherein, the control means comprises one or more valves positioned in the fluid pathway to control fluid flow to and from the filter.

17. An apparatus as claimed in claim 16 wherein, the valves are pinch valves adapted to control the flow of fluid through a pipe in the fluid pathway by compressing the outside of a conduit.

18. An apparatus as claimed in claim 17 wherein, the pinch valve is a solenoid pinch valve.

19. An apparatus as claimed in claim 16 wherein, the at least one valve is controllable so as to trap the cell preservative fluid in the filter such that the cells are immersed in the cell collection fluid prior to their further transportation along the fluid pathway.

20-32. (canceled)

33. A method for collecting cells from a fluid sample, the method comprising the steps of:
controllably pumping a fluid through a fluid pathway which contains a filter; and
filtering the fluid to collect cells of a predetermined size;
wherein pumping the fluid comprises
a first stage in which the fluid from the filter is drawn along the fluid pathway to pass the sample through the filter in a first direction to collect cells on the filter; and
a second stage in which a cell preservative fluid is pumped through the filter in a second direction to remove cells from the filter for collection.

34-53. (canceled)
